(19) United States
(12) Patent Application Publication
(10) Pub. No.: US 2006/0131196 A1
(43) Pub. Date: Jun. 22, 2006

(54) Carrier for a sample chamber, especially for the cryopreservation of biological samples

(76) Inventors: Gunter Fuhr, Berlin (DE); Heiko Zimmermann, Taunus (DE)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(21) Appl. No.: 10/532,281

(22) PCT Filed: Nov. 5, 2003
(86) PCT No.: PCT/EP03/12356

§ 371 Date: Oct. 24, 2005

(30) Foreign Application Priority Data
Nov. 6, 2002 (DE) 102 51 722.3

(51) Int. Cl. A61B 19/02; B01L 3/02; C12N 1/04; C12M 1/00

(52) U.S. Cl. 206/438; 435/307.1; 435/305.3; 435/260; 422/100

(57) ABSTRACT

A carrier (10) for at least one specimen chamber (11), in particular for cryoconservation of biological specimens is described, comprising a mounting frame (20) which is set up for positioning the specimen chamber (11) and has a first frame part (30) and a second frame part (40) which can be detachably assembled with connecting elements (50) and are in contact on the side faces (31, 41) in the assembled state, wherein the specimen chamber (11) can be secured between the side faces (31, 41).

This invention relates to a carrier for at least one specimen chamber for low-temperature storage (also called cryostorage or cryopreservation) of biological suspension specimens, a cryostorage device for suspended biological cells, cell groups or cell constituents and methods of cryostorage.

Cryostorage of biological specimens has numerous applications directed at long-term storage of the specimens while maintaining their vitality. The devices used for this purpose are adapted to the particular specimens and the requirements in specimen handling. In transplantation medicine, for example, organs can be stored at low temperatures, or in transfusion medicine, donor blood can be stored at low temperatures. In these traditional applications of cryostorage, it is usually important to arrange the largest possible specimen volume in the smallest possible amount of space. For example, donor blood is stored in blood bags, which can be packed densely in a blood bank. The blood bags are provided with marking (e.g., a barcode) to permit specific and error-free removal of blood from the blood bank. These techniques are limited to use with relatively large specimens (milliliter to liter range).

In cell biology, however, and to an increasing extent also in molecular biotechnology there is a need for storage and optionally processing biological specimens in extremely small quantities (single cells, cell groups or cell constituents) in a deep-frozen state. For example, EP 804 073 describes the cryoprocessing of individual cells. Cells in a deep frozen state are attached to a substrate stored and optionally processed. This technique has the disadvantage that storage of cells attached to substrates may take up a relatively large amount of space, so this makes it difficult to design a cryocell bank. In addition, to avoid contamination, the specimens must be provided with a protective layer, which makes handling of the specimens difficult.

DE 199 21 236 describes the deposition of droplet-shaped suspension specimens on structured substrates for cryoconservation, whereby the ease of handling of the frozen specimens is facilitated. However, the structured substrates may have disadvantages with respect to the specimen loading and removal of specimens. For specimen loading, all the specimen receptacles must be filled using a dispensing device, the operation of which can be very complex. To take samples, the droplet stored in a specimen receptacle must be completely thawed even if only a portion of the droplet is needed.

Use of cryospecimen chambers (e.g., in the form of capillaries or thin-wall tubes) is known as an alternative to deposition of specimens on substrates. The specimen chambers form an at least partially closed container and therefore provide better protection for the specimens. However, one general disadvantage of the cryospecimen chambers is that thawing of the specimen in the cryochamber in the meantime, possibly unintentional, cannot be detected. The miniaturized specimen chambers or the specimens in them do not undergo any significant change in shape when thawed, as would be the case with a blood bag in a blood bank, for example. Therefore, the quality of the cryoconservation in specimen chambers can usually be monitored only with complex technical assistance.

WO 02/46719 describes a cryostorage device having at least one tubular or pad-shaped specimen chamber which is attached to a data memory. The attachment may be accomplished in particular with at mounting frame on an encapsulation of the data memory. A schematic enlarged side view of a mounting frame traditionally used (e.g., according to WO 02/46719) is illustrated in FIG. 14. The mounting frame 20′ forms a carrier 10′ for tubular specimen chambers 11′ having a circular cross section and supported in receptacle elements 21′. The receptacle elements 21′ are formed as through-holes also having a circular cross section in the mounting frame 20′. With this design the specimen chambers 11′ can easily be threaded into the mounting frame 20′. However, one disadvantage is the low stability of the mount. The specimen chambers can slip or become stuck in the mounting frame. Special measures are necessary for the fixation. In addition, the specimen chambers must be sealed at their free ends. Capillary specimen chambers for cryoconservation are also described in WO 99/20104. The specimen chambers are placed on a cooling body, which results in the same disadvantages as those with the technique according to WO 02/46719.

The object of this invention is to provide an improved cryostorage device with which the disadvantages of conventional cryostorage devices can be overcome and which should permit in particular a stable and reliable mounting of specimen chambers (e.g., for a plurality of suspended cells, cell groups or cell constituents) and a selective partial removal of specimen constituents. The object of this invention is also to provide an improved method for cryostorage.

These objects are achieved with the features according to Patent claims 1, 13 or 15. Advantageous embodiments and applications of this invention are derived from the dependent claims.

A basic idea of this invention is to provide a carrier for at least one specimen chamber in the form of a mounting frame which has at least two frame parts that are in mutual contact laterally in the assembled state with a specimen chamber inserted between them and secured immobiley in relation to the mounting frame. The frame parts may be assembled detachably with connecting elements along their side faces. This advantageously permits a stable means of securing specimen chambers, preferably in the form of a tube, a straw, a pipe or a capillary in the mounting frame. The specimen chambers can be sealed automatically at the ends by pinching them.

By profiling on at least one of the side faces, receptacle elements are formed which allow a minimal clearance to accommodate the wall material of the specimen chamber, e.g., in the form of a slot, in the assembled state of the frame parts. According to a first embodiment of this invention, one of the mutually connecting side faces of the frame parts is flat while the other side face has profiling to form the receptacle elements. This design may have advantages with respect to a simplified structure of the mounting frame. As an alternative, both side faces have recesses facing one another, forming the receptacle elements in the assembled state of the frame parts. This design may be advantageous for error-free assembly of the frame parts.

When the carrier is designed so that the through-holes have an oval or slot-shaped cross section, the specimen chamber inserted, usually having a circular cross section, can be compressed or pinched together. Due to the deformation of the specimen chamber on the receptacle elements, tapered chamber regions are formed, representing intended breaking points for simplified removal of sections of specimen. Furthermore, it is readily possible advantageously to monitor whether cryo conditions are maintained for selectively removed chamber sections due to the fact that the compressed ends of the specimen chamber sections would be deformed if there had been an unintentional thawing in the meantime.

The mounting frame used according to this invention is preferably made of a plastic which is elastically deformable at room temperature, e.g., PE, TPX, polysulfone. The elasticity of the frame material may also simplify the assembly of the frame parts with the at least one specimen chamber.

According to another advantageous design, the frame parts are provided with connecting elements with which the frame elements can be detachably joined together along the profiled side faces. The connecting elements offer the advantage that the frame parts can be joined together without additional adjustment aids and in the assembled state they form an autarkic component. If the connecting elements are formed by combinations of webs and grooves, optionally with latching profiles, then the specimen chamber may additionally be kinked on the receptacle element advantageously.

If according to another embodiment of this invention at least one of the frame parts has tension pins on opposite side faces, this may yield advantages for mounting the specimen chambers on the mounting frame. The tension pins may be formed in particular by the connecting elements between the frame parts.

Further advantages for handling the carrier may be obtained when the two frame parts are pivotably joined together on one side. The pivot connection includes for example a hinge or a strap that can be folded over.

According to an especially preferred embodiment of this invention, the holding frame is connected to a data memory device having at least one data memory. This permits storage of data identifying the specimens in the specimen chambers.

Another subject of this invention is a cryostorage device including at least one inventive carrier and at least one specimen chamber made of a flexible elastically deformable material. Preferably elongated tubular or hollow cylindrical specimen chambers are used as the specimen chambers (designed as a tube or capillary, for example).

Due to the use of at least one elongated specimen chamber, a number of advantages are achieved which have effects on specimen loading, monitoring in cryostorage and removal of specimens. Thus specimen chambers used according to this invention can be filled or emptied rapidly. Liquid transport in the specimen chamber may be accomplished under the influence of the capillary forces or an external excess pressure or vacuum. The specimen chambers are miniaturizable and are flexibly adapted to the use conditions. The inlet ends of a number of specimen chambers used at the same time can be adapted with no problem to any arrangements of specimen reservoirs from which specimens are to be taken. The specimen chambers make it possible to seal off the specimens with respect to the environment. The possibility of contamination from the ambient gas phase or liquid phase (e.g., a coolant medium) is ruled out. Specimens can be removed from a specimen chamber used according to this invention by thawing the entire specimen chamber or partial specimens by mechanical separation, preferably directly at the receptacle elements of the mounting frame, without having to thaw the entire specimen chamber. Mechanical separation may even be accomplished under the influence of local heating without resulting in any loss of the specimen provided in the remainder of the specimen chamber.

One subject of this invention is also a method for cryostorage of suspension specimens, wherein such specimens are accommodated under the influence of capillary forces or external compressive forces in at least one specimen chamber made of a flexible, elastically deformable material, the specimen chambers are securely clamped in the inventive carrier and transferred together to a cryopreserved state. The inventive cryostorage is generally performed at a temperature below room temperature at which long-term storage of specimens without any loss of vitality is possible. The temperature and duration of storage are selected as a function of use. It is especially advantageous to implement this invention in low-temperature storage at temperatures below −100° Celsius and in particular at the temperature of liquid nitrogen.

Further advantages and details of this invention can be seen in the following description of the accompanying drawings, which show:

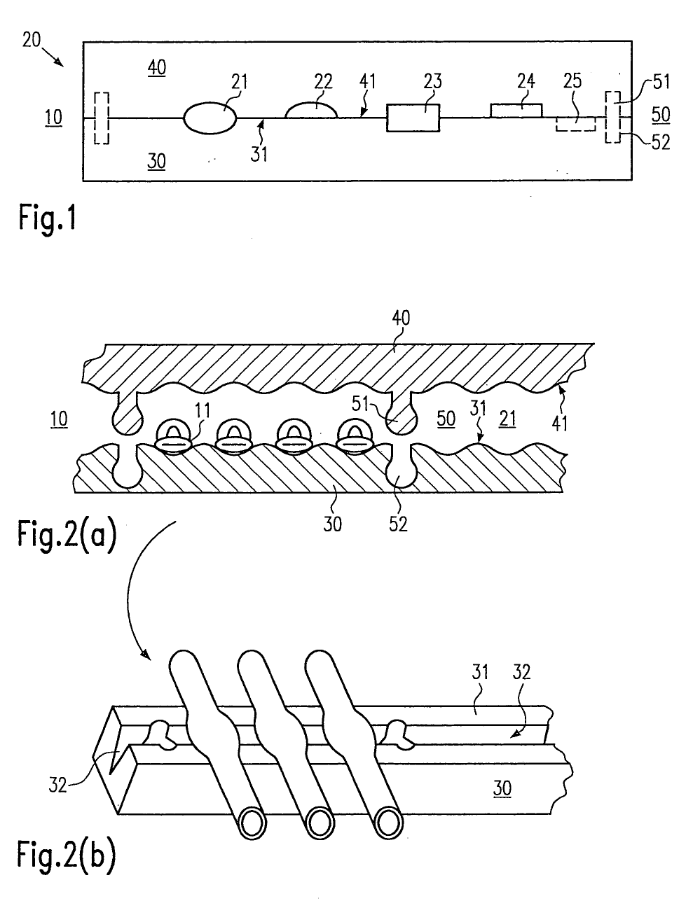

FIGS. 1 through 8: various embodiments of inventive carriers for specimen chambers,

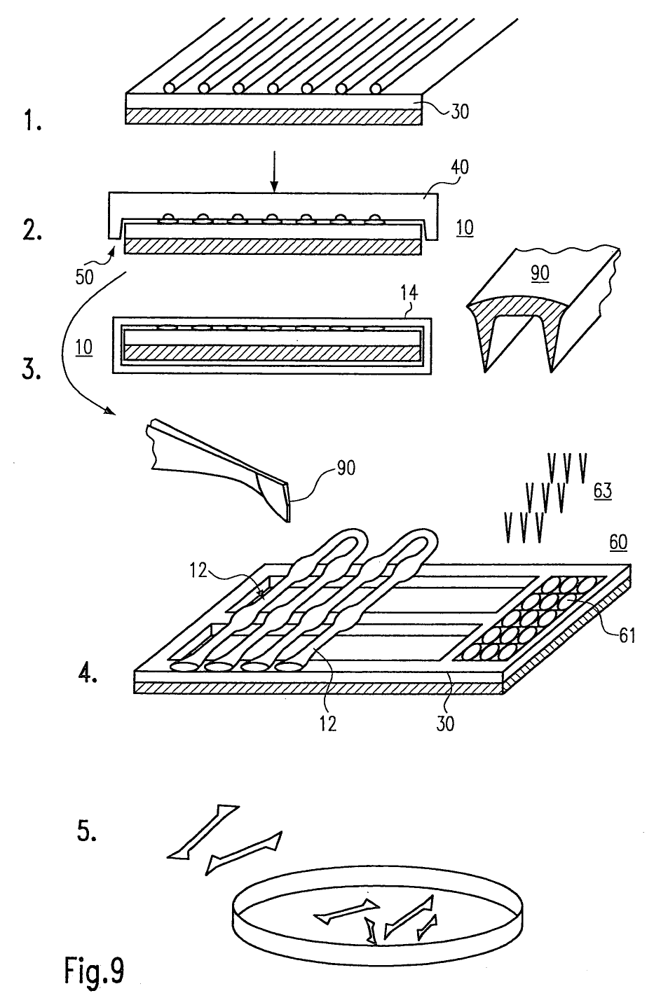

FIG. 9: an illustration of loading the inventive carrier with specimen chambers and removing chamber sections,

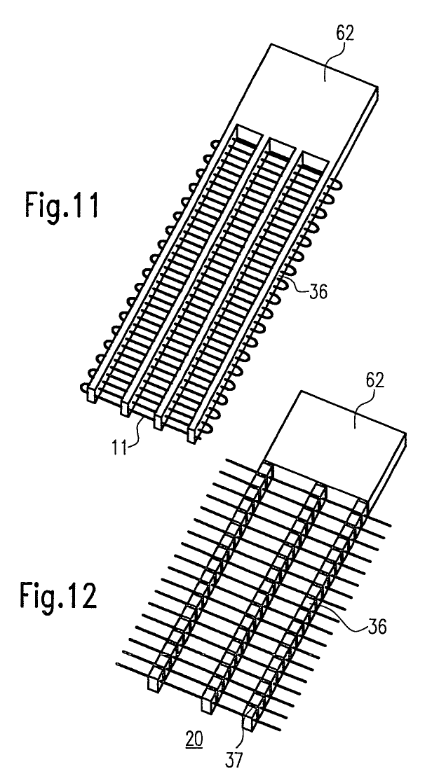

FIGS. 11 and 12: schematic views of other embodiments of inventive carriers for specimen chambers,

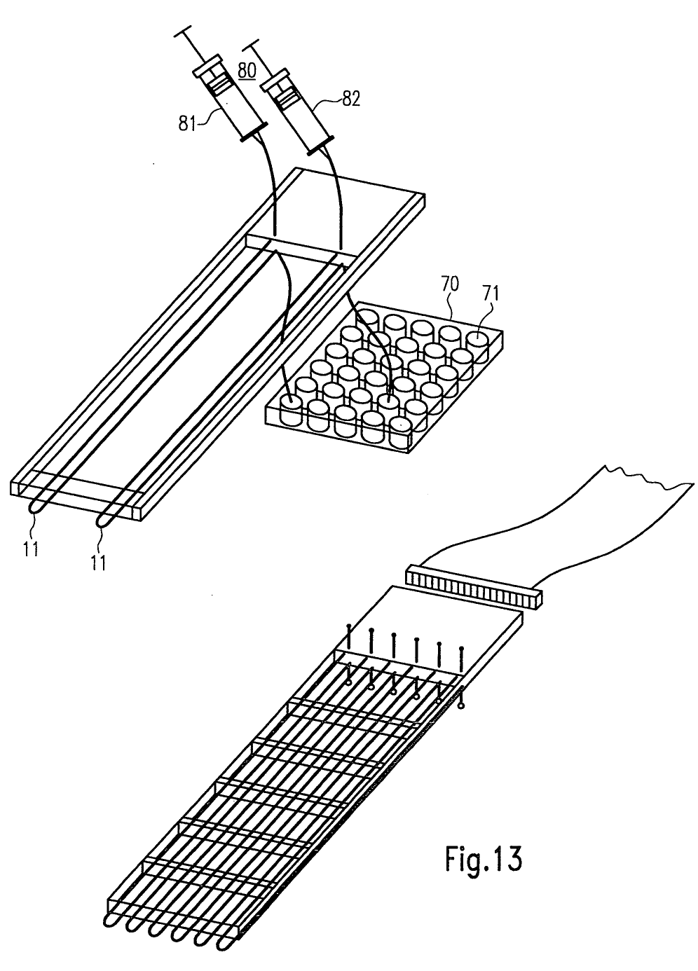

FIG. 13: another illustration of the handling of an inventive cryostorage device, and

FIG. 14: a schematic side view of a conventional mounting frame (state of the art).

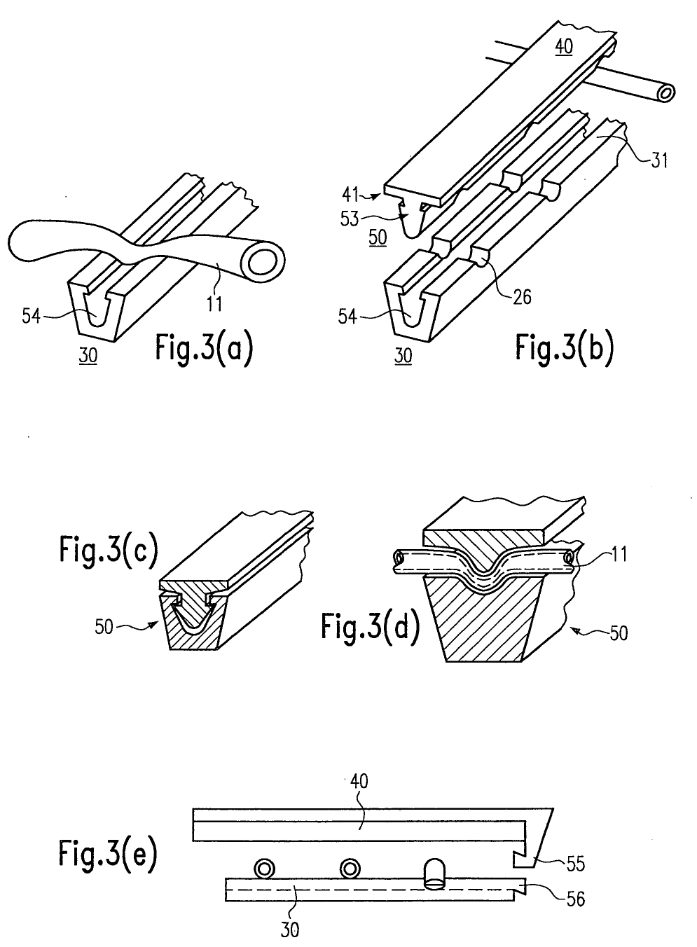

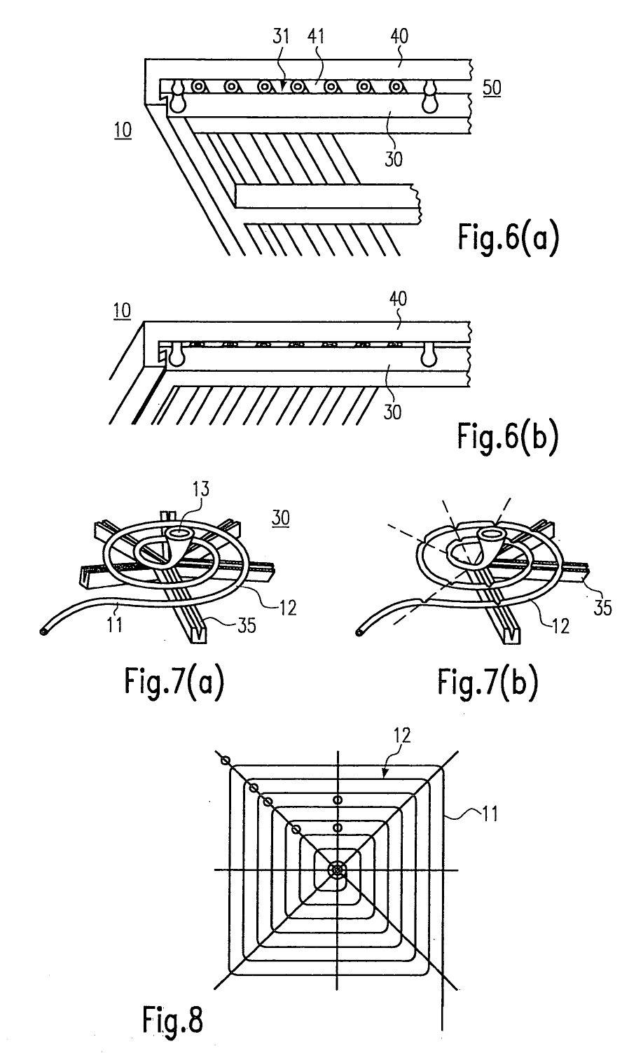

FIG. 1 shows an embodiment of an inventive carrier 10 with different variants of receptacle elements for at least one specimen chamber in a schematic side view. The carrier 10 consists of a mounting frame 20 with a first frame part 30 (also: bottom part 30) and the second frame part 40 (also: cover part 40). The frame parts generally form a circumferential shape, e.g., a rectangular shape which is made up of several frame elements arranged at right angles to one another and/or a star shape with frame elements arranged radially (for example, see FIGS. 7a, b, 8). FIG. 1 shows schematically a detail of the mounting frame 20 for illustration purposes. Each frame part 30, 40 has a side face 31, 41, of which at least one side face 31 and/or 41 may have profiling to form receptacle elements 21 through 25. However, the profiling is not obligatorily provided (see FIG. 6, for example). The frame parts 30, 40 can be assembled with connecting elements 50, e.g., in the form of pins 51 and bushings 52. On one side of the mounting frame, the frame elements 30, 40 can be pivotably joined together. To form the receptacle element 21 having an oval cross section, the two side faces 31, 41 each have profiling with half-oval-shaped recesses facing toward one another. As an alternative, the side face 31 of the bottom part 30, for example, may also be designed to be flat and then only the side face 41 of the cover part 40 is shaped with a recess so that the receptacle element 22 is formed with a half-oval-shaped cross section. Similarly, receptacle elements 23, 24 having triangular, rectangular or slotted cross sections may be formed by profiling the side faces 31, 41 on one or more both sides. The receptacle element 25 represents a special case in which the profiling includes complementary protrusions and recesses in the side faces 31, 41 which permit bending in addition to pinching of the specimen chamber in the carrier. The receptacle element 25 may be part of a connecting element based on a web-and-groove combination. This embodiment of the present invention, which is shown with additional details in FIG. 3, has the advantage that the specimen chamber is sealed at the receptacle elements.

The mounting frame 10 preferably is made of a plastic material that is elastically deformable at room temperature, e.g., PE, TPX, polysulfone. It is preferably produced by an injection molding process. The dimensions of the frame parts 30, 40 are selected as a function of the application and are, for example, in the range of a few cm to a few dm. The receptacle elements, which are generally designed to be rectangular or oval in shape, typically have dimensions which are slightly smaller than the outside dimensions of the specimen chambers.

FIGS. 2a, b show in enlarged form a concrete embodiment of the inventive carrier 10 with additional details. The two frame parts 30, 40 have a corrugated profiling on the side faces 31, 41 to form the receptacle elements 21. The connecting elements 50 are provided at predetermined intervals on the side faces 31, 41 and consist of rod-bushing combinations 51, 52 with latching profiles. The left part of FIG. 2a shows a group of specimen chambers 11, each undeformed (before assembling the frame parts 30, 40) and then also pinched together (after assembling the frame part). FIG. 2b shows the pinched state schematically without the upper frame part 41. The material of the specimen chamber walls that is compressed between the frame parts is advantageously completely pinched so that in addition to the mounting of the specimen chambers, this also yields a local closure and segmentation into subchambers. FIG. 2 also shows that a groove 32 extending in the longitudinal direction of the side faces may be provided on at least one of the side faces 31, 41. The groove 32 forms a trench into which the wall material of the specimen chambers can yield, as illustrated in further details in FIGS. 3a-c.

The various variants of connecting elements 50 are illustrated in FIGS. 3a through 3e. FIGS. 3a through 3e illustrate an embodiment of this invention in which a web 53 having a latching profile is provided as a connecting element 50 on the upper side face 41 of the cover part 40 and on the bottom part 30 a groove 54 having a complementary latching profile is also provided. Furthermore, recesses 26 to form the receptacle elements are provided on the upper and lower side faces 31, 41 at certain intervals (see FIG. 3b). FIG. 3c shows the connecting elements 50 in the engaged state.

A modified design with web and groove-shaped connecting elements 50, but without any latching profiles, is illustrated in FIG. 3d. According to another alternative, the connecting elements 50 may include combinations of hooks 55 and protrusions 56 on the free ends of the frame parts 30, 40 (FIG. 3e).

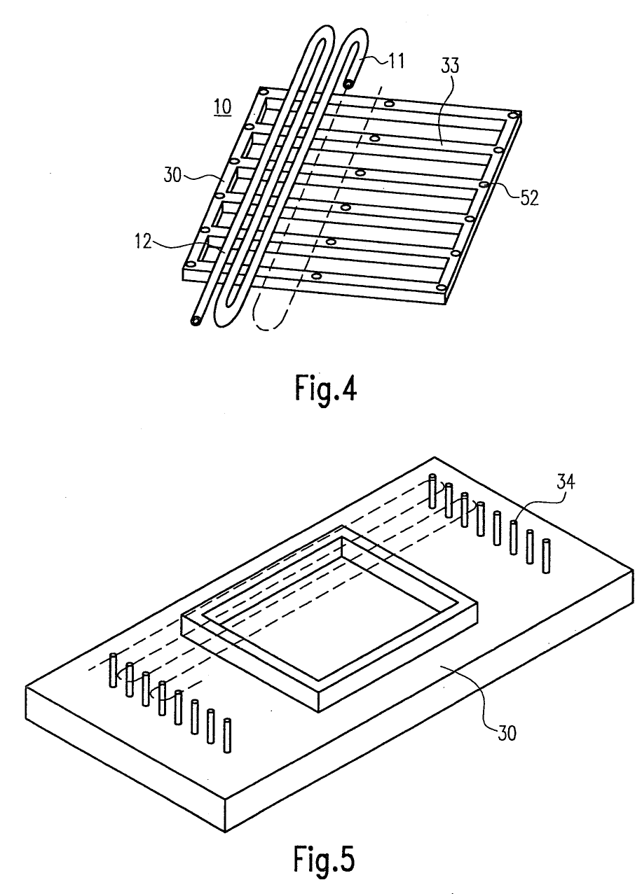

FIG. 4 shows a prospective view from above of the lower frame part 30 of an inventive carrier 10 having a tubular specimen chamber 11. The frame part 30 also includes, in addition to the outer peripheral frame elements, internal frame elements 33 which are designed as webs and also have upper side faces with profiling corresponding to the principles outlined above. Bushings 52 are provided as connecting elements on the outer and inner frame elements. The specimen chamber 11 is laid out in a meandering pattern over the frame part 30. This meandering arrangement has the advantage that a relatively large specimen volume can be accommodated and preserved in an initially continuous specimen chamber. The specimen chamber 11 is divided by the mount on the carrier 10 into individual subchambers or chamber segments which greatly simplifies selective removal of portions of specimens (see FIG. 9). Chamber sections are partial areas of a specimen chamber that run freely between frame elements in the fixated state.

The specimen chambers 11 each consist of a flexible tube or a capillary with an inside diameter that is much smaller than the length of the specimen chamber. For example, the inside diameter may be in the range of 5 μm to 4 mm. The length of the specimen chambers is selected to be in the range of 0.5 cm to 10 dm. The quotient of the cross-sectional diameter and the length of a specimen chamber is preferably less than 1/10. The specimen chambers may be made of any suitable inert and flexible material, e.g., plastic, polymer or metal. Advantageously, miniaturized specimen chambers may also be made of materials having a greater hardness such as glass, semiconductor materials, carbon or ceramics which are elastically deformable at a low wall thickness. The wall material of the specimen chambers may be designed to be impervious or permeable, depending on the desired requirements for cryostorage. In particular, an ion-permeable material may be used such as that known for dialysis tubes. In addition, sensors (e.g., temperature sensors) may be provided in the wall material of the specimen chambers or on the surface thereof. The specimen chambers may be designed with a diameter that is constant over their length, corresponding to the diameter of the inlet and outlet ends (or inlet and outlet openings) of the specimen chamber. In modified embodiments of this invention, a variation of the inside diameter is provided along the length of the specimen chambers.

According to FIG. 5, tension pins 34 may be provided on the frame part 30. The tension pins 34 extend perpendicularly upward from the upper side face 31 of the bottom part 30 and are aligned according to the position of the receptacle elements. The tension pins 34 simplify the layout of the meandering form of the specimen chamber. As an alternative, a separate assembly device may be provided according to this invention wherein the frame part 30 to be loaded can be inserted into the assembly device, which carries the tension pins.

FIGS. 6a and 6b show details of an inventive carrier 10 with the specimen chambers 11 inserted into it before and after assembly of the frame parts 30, 40. In this embodiment of this invention, the side faces 31, 41 are flat and are formed without any profiling. After positioning the specimen chamber 11 (e.g., with tension pins according to FIG. 5), the upper frame part 40 is placed on the specimen chambers 11. By compressing the frame parts that have previously been separated loosely, the connecting elements 50 intermesh. The assembled state is secured by the latching profiles of the connecting elements 50 so that the specimen chambers 11 are arranged to be immovable in respect to the carrier 11 with pinched sections (FIG. 6b).

FIGS. 7a, 7b and 8 illustrate an alternative embodiment of the inventive carrier 10 with frame parts 35 that diverge radially. The specimen chamber 11 is designed in a helical pattern on the frame elements 35 of the lower frame part 30 (upper frame part not shown). Longer chamber sections 12 are formed between the frame elements 35 with an increase in distance from the center. The specimen chamber 11 has a funnel-shaped enlargement 13 at one end in the center of the carrier; it protrudes upward out of the plane of the frame and is used for loading specimen.

FIG. 7a shows the out-laid specimen chamber 11 before assembling the carrier. After assembling the carrier and after the freezing process, this yields the illustration shown in FIG. 7b. Between the chamber sections 12, the specimen chamber 11 is pinched together at the frame elements 35. As an alternative to the spiral arrangement, according to FIG. 8 the specimen chamber 11 may be laid out in a rectangular pattern, resulting in linear chamber sections 12.

FIG. 9 illustrates schematically an advantageous sequence in handling the inventive cryostorage devices. First the specimen chamber(s) is/are loaded. Suspension specimens are accommodated in the specimen chambers by exertion of a vacuum or under the influence of capillary forces, transferring them from storage containers, e.g., the wells of a titer plate. According to steps 1 and 2, the filled specimen chambers 11 are laid out between the frame parts and pressed together by analogy with FIG. 6. In this design, the connecting elements 50 consist of lateral hooks of the upper frame part 40. According to step 3, an additional sealing of the composite structure consisting of the specimen chambers and the carrier may be sealed, e.g., with film sheathing 14. In this state, the freezing operation is performed in this state and the specimens are stored in the frozen state (cryoconservation). The cryostorage device is transferred to an environment with a reduced temperature. This environment is obtained for example in a cryocontainer with liquid nitrogen or with gaseous nitrogen at T=−120° C.

For removing specimens by separating chamber sections 12 after or during cryoconservation, according to step 4 the desired chamber section are mechanically severed with a cutting tool 90. The cutting tool may be designed as single-cut or double-cut (see insert). The chamber sections 12 are cut off directly adjacent to the receptacle elements on the mounting frame 20. Therefore the ends of the severed chamber sections 12 have a deformation deviating from the circular cross-sectional shape, as indicated schematically for step 5. The frozen chamber sections may be stored further in the cryopreserved state in a dish, for example.

The deformation at the ends of the chamber sections is preferably retained as long as the cryopreserved state is maintained.

As an alternative, specimens may be removed by thawing the entire cryostorage device and removing the contents of at least one specimen chamber. For example, this may be done by applying a vacuum at one of the ends of the at least one specimen chamber.

At step 4 in FIG. 9, another important aspect of this invention is illustrated. Advantageously the carrier 10 may be connected to a data memory device 60. The data memory device 60 contains at least one data memory 61, preferably situated in a housing 62 (see FIG. 11). The data memory 61 has an interface for at least temporarily accommodating a plug connection 63 with electric connecting lines by which the data memory 61 can be connected to a control device (not shown).

An essentially known memory chip may be used as the data memory 61 in which any desired data may be stored electrically or magnetically, depending on the application. For example a so-called flash memory may be used. An optical memory may also be provided in the data memory device 60. Data for characterizing the suspension specimens included is entered into the data memory. In the case of medical applications, for example, this data could include identification data for determining the donor and the types of suspended cells, additional features of the donor such as blood group, relevant literature data on the suspended cell types, if applicable, and measurement data and/or image data already available, if applicable. The image data would include, for example, light microscopic images or electron microscopic images of suspended cells used for donor-specific characterization of the cells and later comparison thereof. The saved data is retrieved and/or supplemented during cryostorage.

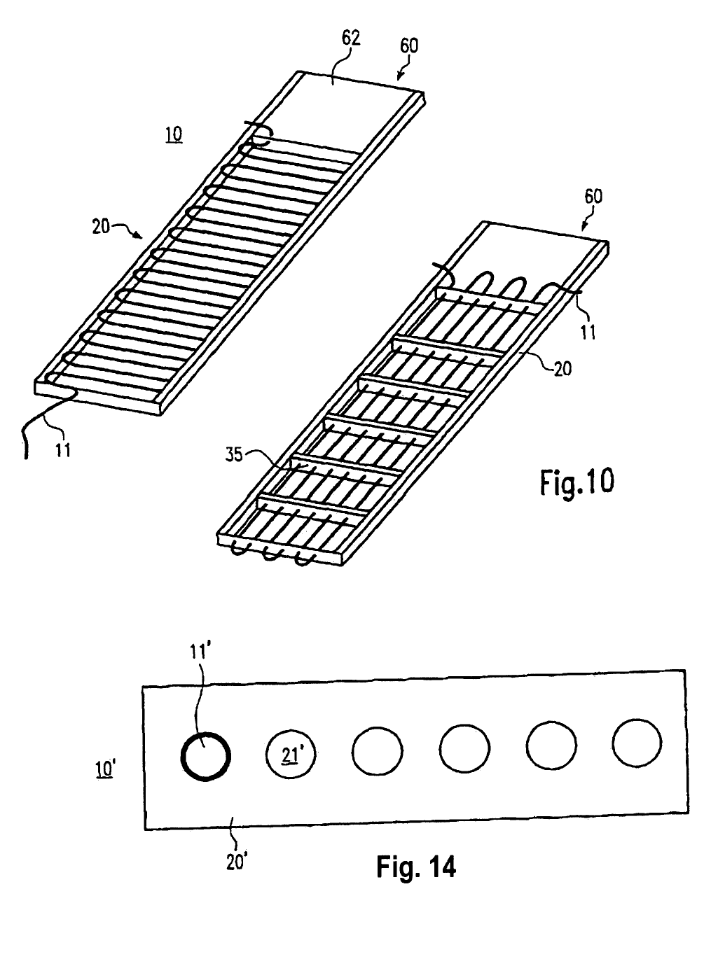

Alternative designs of the inventive carrier 10 with the mounting frame 20 and the data memory device 60 are illustrated in FIGS. 10 through 12. The housing 62 of the data memory device 60 is preferably designed in one piece with the material of the mounting frame 10. The mounting frame 20 is formed as a closed frame by peripheral frame elements according to FIG. 10. Alternatively, according to FIGS. 11 and 12, rod-shaped frame elements 36 are provided, extending from the housing 62 of the data memory. Each frame element 36 includes a number of accommodating elements in which the at least one specimen chamber 11 is secured. For example, a single specimen chamber may be laid out in a meandering pattern through all the frame elements 36 and secured in the laid out or chucked state. Alternatively, multiple separate specimen chambers may also be provided, as illustrated in FIG. 12. The mounting frame 20 may be divisible, e.g., in the form of intended breaking points 37 to separate parts of the specimen chamber(s).

FIG. 13 illustrates one embodiment of this invention in which two specimen chambers 11 are provided and are loaded with various suspension specimens. In practice, the number of specimen chambers belonging to a cryostorage device may be much greater and may be in particular selected as a function of the design of the specimen reservoir 70. For example, the specimen reservoir 70 is formed by a microtiter plate or a nanotiter plate having a plurality of compartments 71. The inlet openings of the specimen chambers 11 each protrude into one of the compartments.

For loading the specimen chambers, the frame parts that are illustrated schematically have not yet been assembled. The suspension specimens are drawn into the specimen chambers. For parallel loading, the suction device 80 includes multiple liquid pipettes 81, 82, . . . according to the number of specimen chambers to be loaded. Alternatively, however, the loading of the specimen chambers may also be performed sequentially with a single liquid pipette 81.

After loading, the frame parts are assembled to secure the specimen chamber(s). The ends of the specimen chambers leading at one end to the specimen reservoir 70 and at the other end to the suction device 80 are cut off. This state is diagrammed schematically in FIG. 13 (bottom). The sealed ends are optionally freely accessible for later access to the contents of the specimen chamber.

The features of this invention as disclosed in the preceding description, the claims and the drawings may be important either individually or in combination for the implementation of this invention in its various embodiments.

1. A carrier for at least one specimen chamber, in particular for cryoconservation of biological specimens, comprising
a mounting frame for positioning the specimen chamber, wherein the mounting frame has a first frame part and a second frame part which can be joined together detachably using connecting elements and which come in contact on side faces of the frame parts in the assembled state, wherein
the specimen chamber can be secured between the side faces of the mounting frame, so that the specimen chamber is immovable relative to the mounting frame.

2. The carrier according to claim 1, wherein one of the side faces is flat and the other side face has a profiling so that receptacle elements are formed between the side faces, and the specimen chamber can be secured in these receptacle elements when the mounting frame is assembled.

3. The carrier according to claim 1, wherein both side faces have profiling so that receptacle elements are formed between the side faces and the specimen chamber can be secured in the receptacle elements when the mounting frame is in the assembled state.

4. The carrier according to claim 1, wherein the receptacle elements have an oval, triangular, rectangular or slotted cross section.

5. The carrier according to claim 1, wherein the connecting elements comprise fitting webs, grooves, pins, bushings, or combination thereof, on the side faces.

6. The carrier according to claim 5, wherein the webs, grooves, pins, bushings, or combination thereof, have latching profiles.

7. The carrier according to claim 1, wherein one of the frame parts has tension pins on its outside, each tension pin being aligned with respect to the receptacle elements so that stretching of the specimen chamber on the frame part is made possible.

8. The carrier according to claim 1, wherein the frame parts are pivotably joined together on one side.

9. The carrier according to claim 1, wherein each of the frame parts have outer frame elements which form a rectangular shape from the mounting frame.

10. The carrier according to claim 9, wherein the frame parts are equipped with inner frame elements which also form receptacle elements in the assembled state so that the specimen chamber can be secured in the receptacle elements.

11. The carrier according to claim 1, wherein each of the frame parts consist of frame elements which extend outward radially in a star shape.

12. The carrier according to claim 1, which is connected to a data memory device having at least one data memory.

13. A cryostorage device, comprising a carrier according to claim 1 and at least one specimen chamber of a flexible, elastically deformable material.

14. The cryostorage device according to claim 13, wherein the at least one specimen chamber is in the form of a hollow cylinder, a hollow cone, a pipe, a tube, a channel or a hollow needle.

15. A method for storage of at least one suspension specimen in a low-temperature state, comprising the steps of:
accommodating the at least one suspension specimen in at least one specimen chamber made of a flexible elastically deformable material,
mounting the specimen chamber in a carrier according to claim 1, wherein the specimen chamber is secured between the frame parts and
converting the suspension specimen to a low-temperature state by positioning the carrier with the specimen chamber in a cryomedium.

16. The method according to claim 15, wherein the receptacle of the at least one suspension specimen into which at least one specimen chamber is inserted by immersing the at least one specimen chamber with one inlet end into a specimen reservoir and transferring the suspension specimen under the influence of a vacuum applied to the corresponding outlet end or under the influence of capillary forces.

17. The method according to one of claim 15, wherein at least one partial specimen is detached from the at least one specimen chamber in the low-temperature state by mechanical separation.

18. The method according to claim 17, wherein the mechanical separation comprises cutting off chamber sections of the specimen chamber adjacent to the frame elements of the carrier.
